(19) United States
(12) Patent Application Publication
(10) Pub. No.: US 2026/0027352 A1
(43) Pub. Date: Jan. 29, 2026

(54) MAGNETIC SAFETY MODULE AND METHODOLOGY TO AVOID TRAUMA DURING COCHLEAR IMPLANT INSERTIONS

(71) Applicants: Daniel SCHURZIG, Hannover (DE); Max FRÖLICH, Hannover (DE); Keno HÜBNER, Innsbruck (AT); Masoud Zoka ASSADI, Innsbruck (AT); Alain Brito SOLANO, Innsbruck (AT); Sarah VORMELCHER, Hannover (DE); Timo BRÄCKER, Celle (DE); MED-EL Elektromedizinische Geraete GmbH, Innsbruck (AT)

(72) Inventors: Daniel SCHURZIG, Hannover (DE); Max FRÖLICH, Hannover (DE); Keno HÜBNER, Innsbruck (AT); Masoud Zoka ASSADI, Innsbruck (AT); Alain Brito SOLANO, Innsbruck (AT); Sarah VORMELCHER, Hannover (DE); Timo BRÄCKER, Celle (DE)

(21) Appl. No.: 19/108,714

(22) PCT Filed: Sep. 1, 2023
(86) PCT No.: PCT/US2023/031840

§ 371 Date: Mar. 4, 2025

Related U.S. Application Data
(60) Provisional application No. 63/404,057, filed on Sep. 6, 2022.

(51) Int. Cl. A61N 1/05; A61N 1/36; A61N 1/375

(52) U.S. Cl. CPC A61N 1/0541; A61N 1/36038; A61N 1/375

(57) ABSTRACT

A system and method for inserting a cochlear implant electrode into a cochlea. A first magnet is fastened to a cochlear implant electrode. A second magnet is coupled, via magnetic attraction, to the first. A force is placed on the second magnet to push the first magnet, and thus also the cochlear implant electrode, into the cochlea. If a threshold force is exceeded, the first magnet and second magnet will separate, preventing a force on the second magnet from causing insertion of the cochlear implant electrode into the cochlea.

### CROSS-REFERENCE TO RELATED APPLICATIONS

This application claims priority from U.S. provisional patent application Ser. No. 63/404,057, filed Sep. 6, 2022, entitled “Magnetic Safety Module and Methodology to Avoid Trauma during Cochlear Implant Insertions,” which is hereby incorporated herein by reference in its entirety.

### TECHNICAL FIELD

The present invention relates to cochlear implants, and more particularly, to systems and methodologies for inserting a cochlear implant electrode array into the cochlea.

### BACKGROUND ART

Unlike conventional hearing aids that just apply an amplified and modified sound signal; a cochlear implant is based on direct electrical stimulation of the acoustic nerve. Typically, a cochlear implant stimulates neural structures in the inner ear electrically in such a way that hearing impressions most similar to normal hearing is obtained.

A normal ear transmits sounds as shown in FIG. 1 through the outer ear 101 to the tympanic membrane (eardrum) 102, which moves the bones of the middle ear 103 (malleus, incus, and stapes) that vibrate the oval window of the cochlea 104. The cochlea 104 is a long narrow duct wound spirally about its axis for approximately two and a half turns. It includes an upper channel known as the scala vestibuli and a lower channel known as the scala tympani, which are connected by the cochlear duct. The cochlea 104 forms an upright spiraling cone with a center called the modiolus where the spiral ganglion cells of the acoustic nerve 113 reside. In response to received sounds transmitted by the middle ear 103, the fluid-filled cochlea 104 functions as a transducer to generate electric pulses which are transmitted to the cochlear nerve 113, and ultimately to the brain.

A typical cochlear prosthesis may include two parts: the audio processor 111 and the implanted stimulator 108. The audio processor 111 typically includes a microphone, a power supply (batteries) for the overall system and a processor that is used to perform signal processing of the acoustic signal to extract the stimulation parameters. The audio processor 111 may be an external behind-the-ear (BTE-) device, may be a single unit that integrates the processor, battery pack and coil or may be implantable.

The stimulator 108 generates the stimulation patterns (based on the extracted audio information) that is provided via an electrode. More particularly, the patterns are sent through an electrode lead 109 to an electrode array 110 implanted through the round window into the cochlear. Typically, this electrode array 110 includes multiple electrode contacts on its surface that provide selective stimulation of the cochlea 104. For example, each electrode contact of the cochlear implant is often stimulated with signals within an assigned frequency band based on the organization of the inner ear. The assigned frequency band of an electrode contact is typically based on its placement within the cochlea, with electrode contacts closer to the base of the cochlea generally corresponding to higher frequency bands.

The connection between a BTE audio processor and stimulator is usually established by means of a radio frequency (RF-) link. Note that via the RF-link both stimulation energy and stimulation information are conveyed. Typically, digital data transfer protocols employing bit rates of some hundreds of kBit/s are used.

A surgeon implanting the cochlear prosthesis, upon drilling the mastoidectomy and opening the round window, has to insert the cochlear implant electrode array through the round window into the cochlea. Deep insertions and accordingly high insertion angles can be beneficial for patients without functional hearing who exclusively hear with their cochlear implant (electric only) See A. Buechner, et al., “Investigation of the effect of cochlear implant electrode length on speech comprehension in quiet and noise compared with the results with users of electro-acoustic-stimulation, a retrospective analysis,” PLoS One 12(5), p. e0174900, 2017 and B. P. O'Connell, et al., “Electrode location and angular insertion depth are predictors of audiologic outcomes in cochlear implantation,” Otol Neurotol 37(8), pp. 1016-23, 2016a which are hereby both incorporate by reference. This is not the case for patients with substantial residual hearing in the low frequency region: for these patients the preservation of this residual hearing is very important. If enough hearing is preserved after implantation, these patients can then use an Electrical Acoustic Stimulation (EAS) system where the low frequencies are stimulated acoustically (e.g. by a conventional hearing aid) and electric stimulation (e.g., by a cochlear implant), is performed only is the basal high frequency region. This kind of stimulation yields higher speech perception results and a more natural hearing impression. Unfortunately, the chance of preserving hearing decreases with increasing insertion depths, (see M. C. Suhling et al., “The impact of electrode array length on hearing preservation in cochlear implantation,” Otol Neurotol 37(8), pp. 1006-15, 2016, which is hereby incorporated by reference) which is why surgeons developed the so-called soft surgical technique. See E. Lehnhardt, “Intracochlear placement of cochlear implant electrodes in soft surgery technique [in German],” HNO 41(7), pp. 356-9, 1993, which is hereby incorporated by reference. Part of this technique is to stop the insertion at the point of first resistance, i.e. at the point at which insertion forces increase to an extent that the surgeon is able to perceive. Alternatively, surgeons could use robotic insertion tools with force sensors to detect this increase in insertion forces. See D. Schurzig et al., “Design of a Tool Integrating Force Sensing With Automated Insertion in Cochlear Implantation,” IEEE ASME Trans Mechatron 17(2), pp. 381-89, 2012, which is hereby incorporated by reference. However, both the manual and automated approach aim to detect the point at which insertion forces increase beyond a specific, predefined point. In traditional, manual surgery this is typically the point after which the electrode array no longer slides into the cochlea but starts to get stuck, which is often followed by a buckling at the cochlear base both extra- and intracochlearly.

Unfortunately, the ability to detect the point at which insertion forces increase more rapidly is quite difficult to detect manually, and a substantial amount of variability in between different surgeons can be expected. Furthermore, the use of partially or fully automated systems into the surgical theater is always liked to a variety of regulatory issues.

### SUMMARY OF THE EMBODIMENTS

In accordance with an embodiment of the invention, a method of inserting a cochlear implant electrode comprising an electrode lead and an electrode array into a cochlea of a subject is provided. The method comprises fastening a first magnet to the cochlear implant electrode, coupling, via magnetic attraction, a second magnet to the first magnet, and applying an insertion force to the second magnet so as to cause the cochlear implant electrode to advance into the cochlea. The magnetic attraction between the first and the second magnets define an insertion force threshold which if exceeded by the insertion force causes the first and second magnets to release, preventing the insertion force from causing further insertion of the cochlear implant electrode into the cochlea.

Optionally, the fastening of the first magnet to the electrode lead includes using a clip to attach the first magnet to the electrode lead.

Also optionally, the magnetic attraction is calibrated to provide a desired insertion force threshold. The calibrating optionally includes adding a separation layer between the first magnet and the second magnet. As another option, the calibrating includes selecting the first magnet and/or the second magnet from magnets of varying strength.

Optionally, the method includes grasping a handle attached to the second magnet, whereby the handle is configured to be manipulated by an operator to advance the cochlear implant electrode into the cochlea. In another option the second magnet includes a handle, whereby the handle is configured to be manipulated by an operator to advance the cochlear implant electrode into the cochlea.

In accordance with another embodiment of the invention, a system for inserting a cochlear implant electrode that includes an electrode array into a cochlea of a subject is provided. The system comprises a first magnet, a fastener which attaches the first magnet to the electrode lead, and a second magnet. The second magnet is magnetically attracted to the first magnet such that when an insertion force on the second magnet exceeds a threshold the second magnet separates from the first magnet, limiting further insertion of the cochlear implant electrode into the cochlea.

Optionally, the fastener includes a clip. Also optionally, the magnetic attraction between the first magnet and the second magnet is calibrated to provide a desired insertion force threshold. The calibration optionally includes selecting the first magnet and/or the second magnet from magnets of varying strength.

Also optionally, the system includes a separation layer between the first magnet and the second magnet. Optionally, the system further comprises a handle attached to the second magnet, wherein the handle is configured to be manipulated by an operator to advance the cochlear lead into the cochlea. Alternatively, the second magnet comprises the handle.

Optionally the system further comprises a plunger attached to the second magnet, said plunger configured to move through a barrel. Also optionally, the magnetic attraction corresponds to a maximum allowed insertion force, which if exceeded by the insertion force will cause the first and second magnet to decouple, stopping further insertion of the cochlear implant electrode into the cochlea.

### BRIEF DESCRIPTION OF THE DRAWINGS

The foregoing features of embodiments will be more readily understood by reference to the following detailed description, taken with reference to the accompanying drawings, in which:

FIG. 1 shows a conventional cochlear prosthesis;

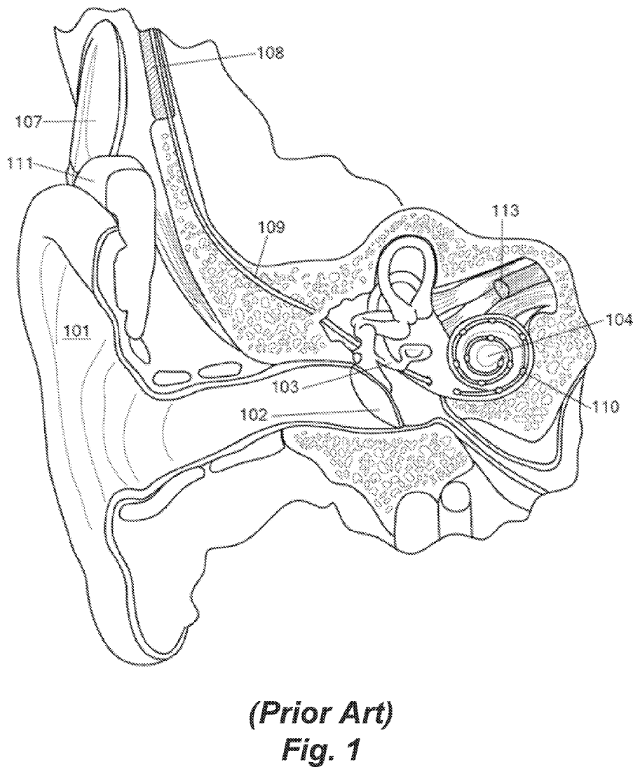

FIGS. 2A and 2B depict one embodiment of a magnetic safety module, in accordance with an embodiment of the invention;

FIGS. 3A and 3B depict the magnetic safety module of FIG. 2 attached to a cochlear implant electrode and force instrument, in accordance with an embodiment of the invention; and

FIGS. 4A and 4B depict the magnetic safety module of FIG. 2 further including use of a plunger to provide the insertion force, in accordance with an embodiment of the invention.

### DETAILED DESCRIPTION OF SPECIFIC EMBODIMENTS

In illustrative embodiments, a system and method is provided for inserting a cochlear implant electrode having an electrode array into a cochlea. The system and method may utilize a pair of magnets which separate when a force greater than a threshold is applied. The separation removes the force from being placed on to the cochlear implant electrode. Details are discussed below.

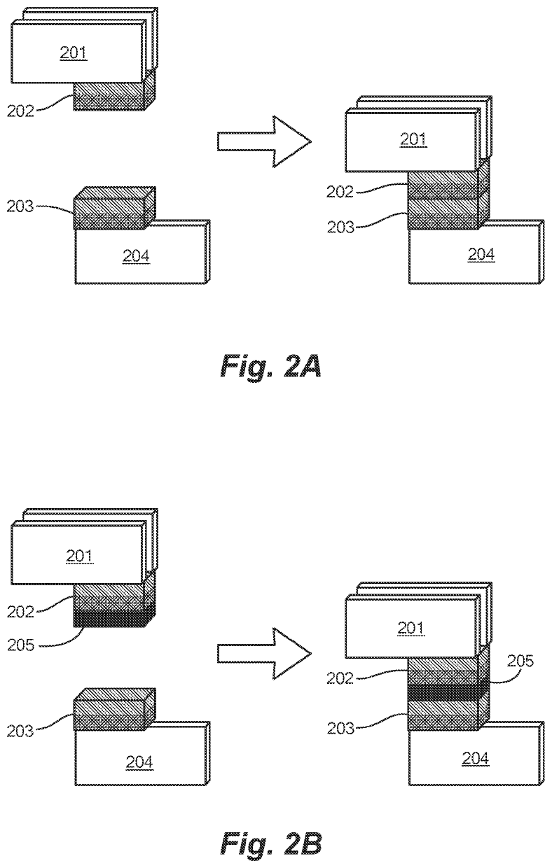

FIG. 2A depicts one embodiment of a magnetic safety module in accordance with an embodiment of the invention. Displayed on the left side is fastener 201 fixed to magnet 202. Fastener 201 may attach, for example, to an electrode lead, and thus as the fastener 201 and/or magnet 202 are forced in a direction, the electrode lead would be forced in said direction as well. In FIG. 2A, magnet 203 is affixed to a handle 204. The handle 204 is designed to receive a force and apply it to the magnet 203. When the two magnets 202, 203 are connected, as shown on the right side of FIG. 2A, a downward force applied to the handle 204 will either apply a corresponding downward force to fastener 201 or cause the magnets 202, 203 to separate. If the downward force exceeds the magnetic force the magnets, they will separate, and if the force is less than the magnetic force the magnets 202, 203 will both be pulled downward. In various embodiments, the force may be in any direction, in fact, in some embodiments a force in one direction on the magnets may produce a force in a different direction on the cochlear implant electrode.

In various embodiments, one or both of the fastener 201 and handle 204 are the magnets 202, 203. In these embodiments, the magnet 202 is capable of attaching to the cochlear implant electrode and the magnet 203 is capable of functioning as a handle or otherwise accepting a force. Alternatively, the magnets may be arranged differently than shown in FIG. 2A. For example, the magnets may be attached such that they are side by side. In such an embodiment, an operator may notice the magnets begin to slide apart and can lessen the force before the cochlear implant electrode buckles or is stuck.

In FIG. 2B, a magnet separator 205 has been added, which is placed between the magnets 202, 203. The magnet separator 205 changes the force required to separate the magnets by increasing their distance apart. A magnet separator may be affixed to either magnet 202, 203 or held in place by the magnetic force during operation. The magnet separator 205 may be adjustable in size or multiple separators having different sizes may be used to vary the force required for separation required in various situations. To achieve the same goal, one skilled in the art would appreciate that varying the strengths of the magnets would provide varying forces required for separation.

The fastener 201 may be a clip, which would be attached to the electrode lead before it is advanced. The handle 204 and fastener 201 are, preferably, snapped together via magnetic attraction before insertion. The handle 204 is then gripped by something to transfer the insertion force (e.g. a hand or plunger) the magnets will transfer that force to the lead, unless it exceeds a threshold, in which case the magnets would separate.

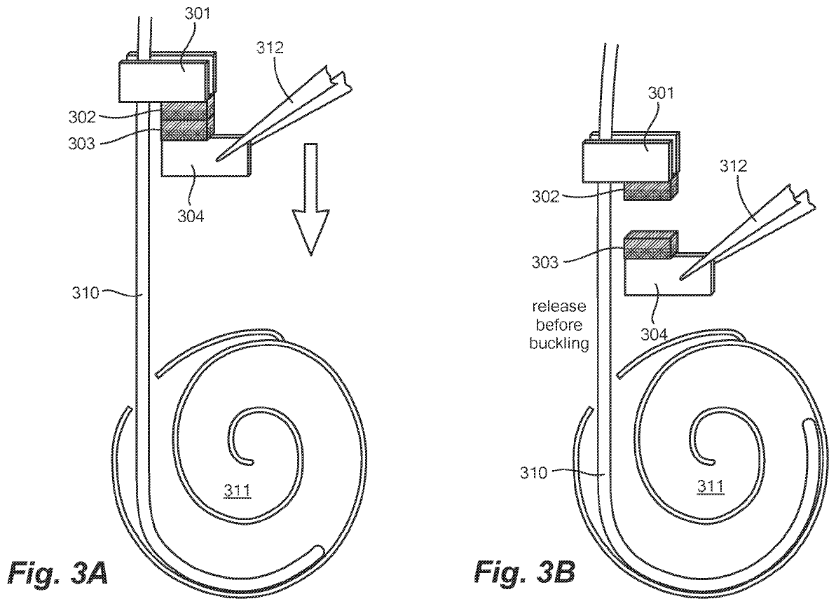

FIGS. 3A and 3B depict the magnetic safety module of FIG. 2 attached to a cochlear implant electrode and force exerting instrument. The cochlear implant electrode 310 is inserted into cochlea 311 as shown. As the tip of the cochlear implant electrode 310 moves into the cochlea, the cochlear implant electrode may buckle or the tip may get stuck. If either of these happen, a larger force will require to be exerted on the handle 304, which would cause the magnets to separate, as shown in FIG. 3B. When the magnets separate the force actor 312 will no longer put a force on the cochlear implant electrode 311. This may be advantageous to help prevent damage to the cochlea where the tip is stuck, or to reduce buckling, allowing the cochlear implant electrode to continue to be pushed toward the cochlea, without having to first remove the cochlear implant electrode. In one embodiment the magnets should be calibrated such that the force holding them together corresponds to the insertion force at which a further insertion of a cochlear implant electrode should be stopped to maintain hearing preservation.

In other embodiments, the force actor 312 directly grasps the lower magnet 303. In such an embodiment the magnet 303 may be shaped like a handle. Similarly, in some embodiments, the top magnet 302 may comprise the fastener 301. The force required to separate the magnets 302, 303 may be calibrated to be a force less than an insertion force that would cause the cochlear implant electrode 310 to buckle. Once the desired separation force is determined, the magnets may be selected at specific strengths or a magnet separator may be used to lessen the attraction force. In other embodiments, the magnets 302, 302 may be varying magnets that could change based on the depth of insertion of the cochlear implant electrode 310. For example, two electro magnets could be used such that as the insertion depth increases, the strength of the magnets decreases allowing better protection of potentially more sensitive parts of the cochlea.

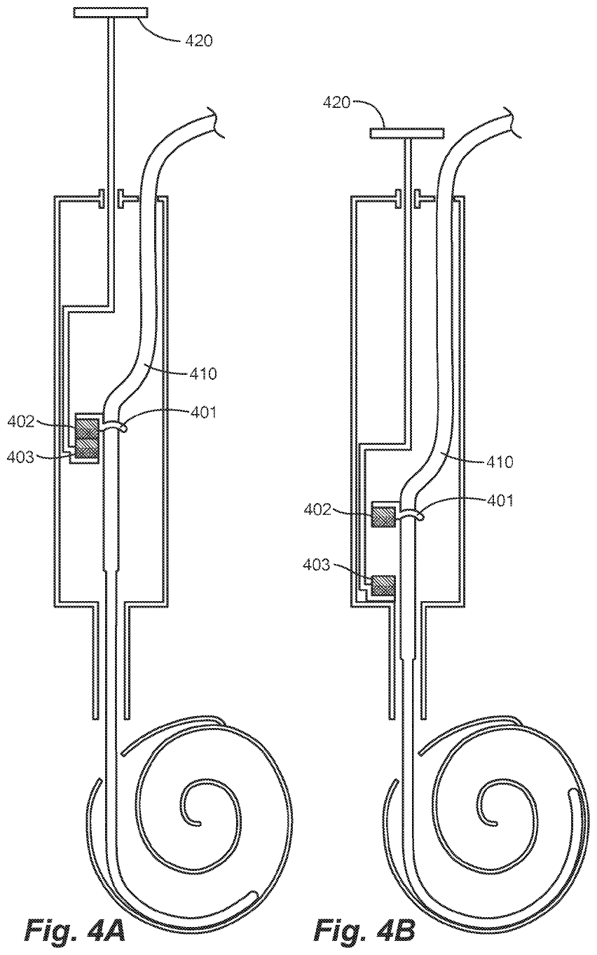

FIGS. 4A and 4B depict the magnetic safety module of FIG. 2 further including use of a plunger to provide the insertion force. In FIG. 4A, the plunger top 420 is shown extended, the cochlear implant electrode 410 is attached to the first magnet 402 via clip 401, and the internal end of the plunger is shown attached to the second magnet 403. The internal end of the plunger may also be attached to a handle, if present. As shown in FIG. 4B, as the plunger was pushed down, the cochlear lead was moved further into the cochlea. However, at some point the insertion force exceeded a threshold, causing the magnets 402, 403 to separate. In some embodiments the plunger provides a more constant direction to the force. Furthermore, through the use of gears or other various methods, a plunger may allow the operator, such as a surgeon or a surgical robot, to move the plunger a distance proportional to the distance the cochlear implant electrode is being inserted, which may aid in sensitivity, speed, or other factors of the insertion.

The embodiments of the invention described above are intended to be merely exemplary; numerous variation and modification will be apparent to those skilled in the art. All such variations and modifications are intended to be within the scope of the present invention ads defined in any appended claims.

What is claimed is:

1. A method of inserting a cochlear implant electrode comprising an electrode lead and an electrode array into a cochlea of a subject, the method comprising:
fastening a first magnet to the electrode lead;
coupling, via magnetic attraction, a second magnet to the first magnet;
applying an insertion force to the second magnet so as to cause the electrode lead to advance into the cochlea,
wherein the magnetic attraction between the first and the second magnets defines an insertion force threshold which if exceeded by the insertion force causes the first and second magnets to release, preventing the insertion force from causing further insertion of the cochlear implant electrode into the cochlea.

2. The method according to claim 1, wherein fastening the first magnet to the electrode lead includes using a clip to attach the first magnet to the electrode lead.

3. The method according to claim 1, further including calibrating the magnetic attraction to provide a desired insertion force threshold.

4. The method according to claim 3, wherein calibrating the magnetic attraction includes adding a separation layer between the first magnet and the second magnet.

5. The method according to claim 3, wherein calibrating the magnetic attraction includes selecting the first magnet and/or the second magnet from magnets of varying strength.

6. The method according to claim 1, further including a handle attached to the second magnet, the method further comprising grasping of the handle whereby the handle is configured to be manipulated by an operator to advance the cochlear implant electrode into the cochlea.

7. The method according to claim 1, wherein the second magnet includes a handle, the method further comprising grasping of the handle whereby the handle is configured to be manipulated by an operator to advance the cochlear implant electrode into the cochlea.

8. The method according to claim 1, further including a plunger configured to move through a barrel, the method further comprising fastening the second magnet to the plunger.

9. The method according to claim 1, wherein the magnetic attraction corresponds to a maximum allowed insertion force, which if exceeded by the insertion force will cause the first and second magnet to decouple, stopping further insertion of the cochlear implant electrode into the cochlea.

10. A system for inserting a cochlear implant electrode comprising an electrode lead and an electrode array into a cochlea of a subject, the system comprising:
a first magnet;
a fastener which attaches the first magnet to the electrode lead;
a second magnet which is magnetically attracted to the first magnet such that when an insertion force on the second magnet exceeds a threshold the second magnet separates from the first magnet, limiting the insertion force from causing further insertion of the cochlear implant electrode into the cochlea.

11. The system according to claim 10, wherein the fastener includes a clip.

12. The system according to claim 10, wherein the magnetic attraction between the first magnet and the second magnet is calibrated to provide a desired insertion force threshold.

13. The system according to claim 10, further comprising a separation layer between the first magnet and the second magnet.

14. The system according to claim 12, wherein calibrating the magnetic attraction includes selecting the first magnet and/or the second magnet from magnets of varying strength.

15. The system according to claim 10, further comprising a handle attached to the second magnet, wherein the handle is configured to be manipulated by an operator to advance the cochlear implant electrode into the cochlea.

16. The system according to claim 10, wherein the second magnet comprises a handle configured to be manipulated by an operator to advance the cochlear implant electrode into the cochlea.

17. The system according to claim 10, further comprising a plunger attached to the second magnet, said plunger configured to move through a barrel.

18. The system according to claim 10, wherein the magnetic attraction corresponds to a maximum allowed insertion force, which if exceeded by the insertion force will cause the first and second magnet to decouple, stopping further insertion of the cochlear implant electrode into the cochlea.
